(19) United States
(12) Patent Application Publication
(10) Pub. No.: US 2013/0316428 A1
(43) Pub. Date: Nov. 28, 2013

(54) Process for the production of fuel gas from municipal solid waste

(71) Applicant: VM PRESS S.R.L., OVADA (IT)

(72) Inventor: Carlo GONELLA, ROCCAGRIMALDA (IT)

(73) Assignee: VM PRESS S.R.L., OVADA (IT)

(21) Appl. No.: 13/899,717

(22) Filed: May 22, 2013

(30) Foreign Application Priority Data
May 25, 2012 (IT) TO2012A000456

(51) Int. Cl. C12P 1/04

(52) U.S. Cl. CPC C12P 1/04
USPC 435/170

(57) ABSTRACT

After separating from solid urban waste an organic fraction containing biological cells, the latter is extruded through a grid having small-bore holes, under a pressure higher than the bursting pressure of the cell membranes, so that most of these are disrupted and a gel of a doughy consistency is produced. The gel is then loaded into a biodigester, where it is readily attacked by bacteria.

The present invention is mainly concerned with the production of fuel gas (biogas) from municipal solid waste (MSW), although the invention can be applied, more generally, also to the disposal of agricultural and industrial waste containing an appreciable fraction of moist, organic material. More particularly, the invention is concerned with a method for improving the efficiency in the production of fuel gas by recycling the organic fraction of MSW.

### BACKGROUND OF THE INVENTION

Municipal solid waste, such as is collected in the dumpsters of the waste disposal services, typically contains, beside inert materials such as paper, plastics, glass and metals, a substantial moist organic fraction, mainly comprising kitchen refuse, rejected fruits and vegetables, liquid remnants of milk and fruit juices, grass, garden waste, and the like. The organic fraction is usually prevalent in farm waste, and industrial refuse (particularly in the food industry) often comprises substantial amounts of fermentable organic material.

It is known to separate the organic fraction from the remaining material by mechanical means (unless the garbage has been sorted beforehand at the kerb), and to subject it to anaerobic fermentation in a biodigester, in order to produce, on the one hand, fuel gas and, on the other hand, a stabilized solid residue that is often usable as a soil conditioner.

Before being fed to the biodigester, the organic fraction is converted to a sludge by mincing, beating and diluting, so that the anaerobic bacteria can more easily spread and attack the organic matter. The nature of the organic matter, the proportion of the dilution water, the thoroughness of the stirring and the temperature changes are some of the more important factors in determining the effectiveness of the gasification of the solid organic matter. In order to assess the effectiveness of conversion of the solid matter into biogas (which essentially consists in a gaseous mixture of methane, carbon dioxide, and small amounts of other gases such as hydrogen, hydrogen sulfide, etc.) it is usual to measure the reduction rate of volatile solids (SV) (which in turn is derived from a measure of the reduction rate of total solids, ST) in a predetermined fermentation time, typically 20 to 30 days. The higher the SV reduction rate, the larger is the production of biogas, and the smaller are the residual solids.

Obviously, it is desired to obtain the largest possible daily production of biogas, and this requires that the gasification of volatile matter is as complete as possible. This target, however, involves a very long dwelling time of the organic matter in the biodigester (typically 20 to 30 days as stated above), so that the bacterial flora can progressively digest even the toughest components of the matter to be fermented.

It is a well-known problem of conventional biodigestion that the minced and stirred organic material tends to separate from the dilution water: on the one hand, a portion of the the material (mostly residual inert materials left in the organic fraction) will settle on the bottom and coalesce in a compact layer; on the other hand, another portion of the material will float to the surface and form a fibrous layer, sometimes called a “hat”. Both the sediments and the hat hinder the progress of the fermentation, not only because they hamper the attack of the anaerobic bacteria, but especially because both the hat and the sediments have to be removed periodically, by a burdensome and time-consuming operation, which involves a considerable down-time for the digester.

In order to prevent or reduce the formation of solid sediments, the organic sludge is generally left in repose for a short time, so that the heavier inert particles can settle down to the bottom. The sludge is then spilled by slow overflow to a tank, while the lightweight particles are retained by a baffle, and is then loaded to the biodigester. However, this step adds to the cost of the process, because it requires considerable additional equipment and lengthens the overall processing time. Moreover, even this step only partly solves the problem. In fact, the beating or whipping step performed to obtain the desired sludge will also crumble down some inert fragments to fine particles, which are dispersed throughout the sludge and become englobed in clots of the organic sludge and are thereby prevented from sinking to the bottom while the sludge is left in repose. However, as the sludge is later fed to the biodigester, the organic clots are progressively dissolved by the attack of the bacterial flora, and the fine particles become free and eventually either sink to the bottom, where they add to the above discussed compact bottom layer, or float to the surface, contributing to the formation of the hat.

### SUMMARY OF THE INVENTION

It is now the main object of the invention to improve the method of production of fuel gas from the organic fraction of municipal solid waste by increasing its efficiency, so that the fermentation and production of biogas is completed more quickly and with higher yield, while the production of solid residue is decreased.

Another object of the invention is to shorten the dwelling time of the solid material in the biodigester for equal effectiveness of the method.

Still another object is to reduce the formation of solid sediments at the bottom of the biodigester, thereby reducing the frequency of shutdowns necessary for breaking up the bottom layer.

The invention attains the above and other objects and advantages, such as will appear from the disclosure below, by a method for producing fuel gas starting from an organic fraction of MSW, having the features recited in claim 1.

Other advantageous features of the invention are set forth in the dependent claims.

### BRIEF DESCRIPTION OF THE DRAWINGS

The invention will now be described in more detail with reference to the attached drawings and a few examples. In the drawings:

FIG. 1 is a partial diagrammatic view, in axial cross-section, of an extrusion press used in the method of the invention;

FIG. 2 is a diagram of a biodigester pilot plant used for testing the method of the invention;

FIG. 3 is a bar chart showing the results of a number of biodigestion tests which were carried out in two plants according to FIG. 2, starting from conventional pulp material; and

FIG. 4 is a bar chart showing the results of a number of biodigestion tests which were carried out in the same plants according to FIG. 2, starting from material that was extruded and gelled according to the concepts of the invention.

### DESCRIPTION OF THE PREFERRED EMBODIMENTS

In developing the invention, the inventors started from the consideration that, in the course of the molecular conversion of the organic waste, the material to be attacked by bacteria, even if minced or crushed, still largely comprise macroscopic fragments, consisting of compact clusters of organic cells, in which the walls of most cells are largely unbroken. Consequently, attacking bacteria first have to break down the cell membranes, which oppose a stronger resistance than the rest of the material, and are especially tough in the case of vegetal cells: this circumstance slows down the process considerably. A further slowdown is caused by the above mentioned tendency of the particles of organic material to separate from the dilution water. Because of this, the material will progressively settle or float, forming increasingly compact layers: these layers, as they dry up, become more and more invulnerable to attack by bacteria.

In contrast to the prior practice of mincing or whisking the moist organic material in grinders and pulpers, such as hammermills, this invention provides that the organic material is squeezed or extruded under a very high pressure through an extrusion grid having small-bore holes, so that the more humid and fragile component is forced to go through the holes and thus become completely disintegrated to a semi-liquid pulp, leaving a substantially dry component upstream of the grid. Because of the high compression, the extrusion or squeezing does not merely mince the material at a macroscopic level, but rather causes an actual diffuse tearing of the cell structure, and bursts open the membranes which normally hold within the cells the semi-liquid organic matter, so that it becomes more readily attacked by the bacteria.

Moreover, the extruded pulp, which largely consists of biological material leaked out from the torn cells, and subjected to the above mentioned high pressure, has the nature of a thick solid-liquid gel, similar to a jam or jelly, having little or no inclination to separate the water content from the solid component. This circumstance makes it possible to feed the gel to the biodigester more smoothly, for instance by pumping, without having to maintain a strong turbulence in order to prevent sedimentation, as in the prior art.

As described below, the inventors have observed that, when the biodigester is fed with an organic fraction which has undergone the above preliminary treatment, the biochemical fermentation reaction starts quickly, yielding a high production of fuel gas while giving rise to only a limited production of solid residue. The gas that is generated has a very good quality and the conversion process is completed in a shorter time span than could be achieved in the prior art, as will be shown by the examples disclosed below.

It is assumed that the increased efficiency of the conversion, and in particular the short time required for starting the fermentation, is due to the fact that the cell contents, which are no longer protected by the cell membranes, are readily attacked by the bacteria.

According to a preferred embodiment of the invention, and with reference to the schematic diagram of FIG. 1, a wet organic fraction (also known as biodegradable municipal waste, or BMW) is obtained from municipal solid waste and is then compressed in successive portions in a cylindrical grid comprising a high-resistance tube 10 that is perforated with small-bore holes 12 and is closed at one end by a wall 14, while its opposite end 16 is open. The organic material is pushed into the open end 16 of tube 10 by a piston 18 driven by a hydraulic cylinder not shown. The pulp emerging through holes 12 runs into an underlying tray (also not shown) as a thick gel, similar to a jam, which does not release free water even after a prolonged settling time, except in negligible amounts.

An extrusion press according to the diagram of FIG. 1, and which is suitable for implementing the method of the invention, is disclosed in documents such as European patent EP-A-1 207 040, entitled “Press for treating solid city waste”.

The extrusion holes preferably have a bore smaller than 12 mm, and even more preferably smaller than 10 mm, ideally a bore of about 8 mm. Since the high extrusion pressure causes a correspondingly high wear of the grid, the holes may be lined with of a hard metal or a ceramic, or the grid itself may be made of a high-resistance material, such as a special steel, e.g. as disclosed in EP-A-1 568 478.

After each compression, a small-volume, dry residue is left within the grid, having a water content reduced to a negligible amount, typically less than 20%. Such a dry residue is then expelled and led to further treatments which do not belong to the inventive method, such as incineration.

A number of tests were carried out upon samples of substantially the same organic fraction, in order to assess the effectiveness of the inventive concepts. The organic fraction was conditioned by conventional pulping in some tests, and by presso-extrusion according to the invention in other tests.

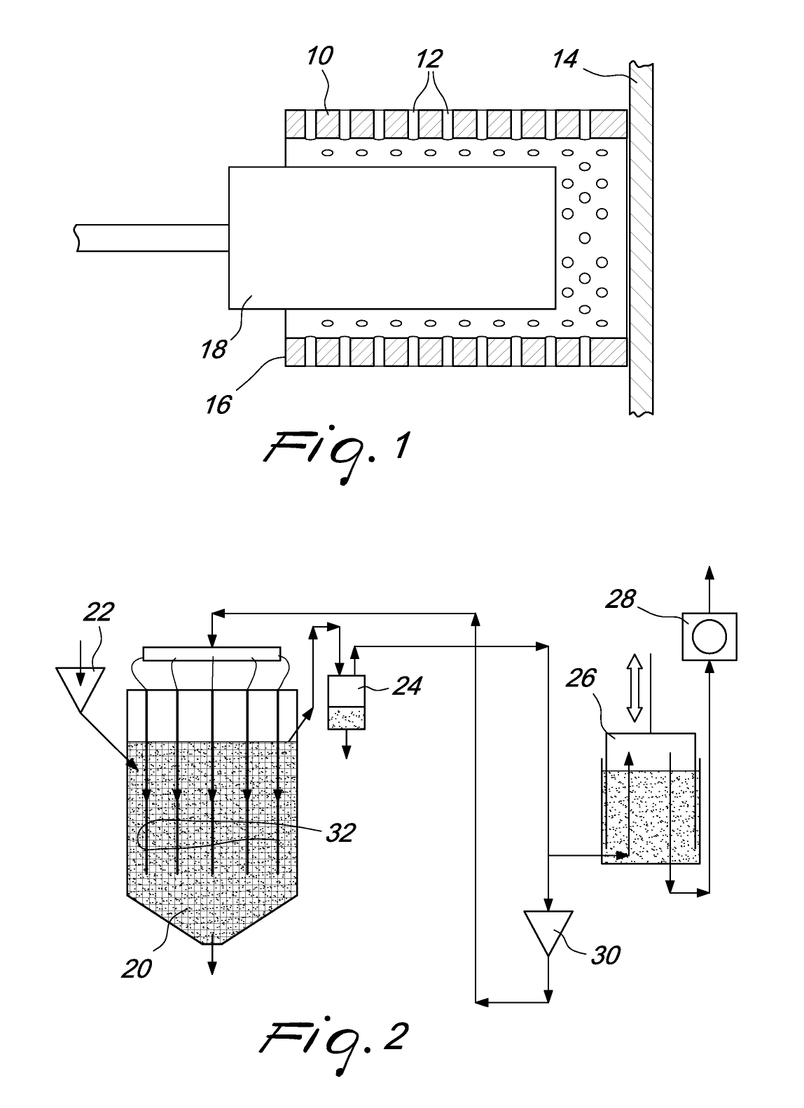

The above-mentioned tests were held at different times with substantially uniform procedure, in two pilot biodigestion plants, each set up as shown in the diagram of FIG. 2. Each plant comprised a biodigester 20 that could be loaded through a hopper 22 and was connected to a bell-shaped gasholder 26 via a condensate remover 24, whereby the generated biogas could be collected. The amount of gas flow was measured by a gas meter 28.

The biodigester 20 itself was a vertical steel cylinder, tapering at its bottom into a cone having an opening for periodically unloading the digestate. The biodigester was surrounded by a serpentine (not shown) for circulation of warm water under control of a thermostatic valve, in order to maintain the biodigester at a constant temperature. Internal pressure and pH were also monitored.

Hopper 22 was loaded with organic material that had been prepared and diluted to the desired condition (either according to the conventional process or according to the inventive process) and the digestate was unloaded at the bottom.

The plant also comprised a compressor 30 for tapping biogas from gasholder 26 and recirculating it to the biodigester, through a rose of vertical nozzles 32. Biogas was thus injected into the biodigester at its bottom and then rose to the top, thereby stirring and mixing the organic material, and preventing it from settling. By so bubbling through the sludge, the injected biogas also had the result of continually disgregating the floating fibrous particles and thus preventing them from mutually coalescing, which otherwise would ultimately lead them to form a so-called “hat”.

The biogas surplus was stored in gasholder 26 and contributed to maintain a stationary pressure in the biodigester. When the gasholder was full, a limit switch (not shown) automatically opened an exhaust valve in order to convey the biogas to gas meter 28.

Several tests of biodigestion were made, both according to the conventional process using pulped BMW and according to the inventive process in which the BMW was extruded to a gelified material. At the beginning of each test, the first load of pre-treated BMW was primed with an inoculum comprising bovine sewage which had been diluted to a concentration of organic matter of 3% in weight. The pre-treated BMW was then loaded in daily portions such that they gave rise to dwelling times of 25 days, with an overall duration of the test of about 50 days. The generated biogas prodotto was measured in gas meter 28, and the dumped digestate was each time weighed. Through all the tests, the biodigester was constantly maintained at a temperature of 40° C., by means of the above-mentioned heated serpentine, under control of a thermometric probe not shown.

Six tests were run using the conventional process, in which the organic fraction (BMW) was minced and mixed in a pulping system comprising a hammer mill known per se, the dilution water being chosen at a different proportion for each test, so that the solid percentage (ST) was 4%, 8%, and 10% in weight of the total weight, respectively.

From the data measured in the several tests, the reduction rate of the total solids (RS %) was computed, and this value was linked to the volume of collected biogas and to the weight of unloaded digestate, thereby yielding the reduction rate of volatile solids (RV %). The latter value is a measure of the degree of conversion of volatile solids into biogas, and is therefore a measure of the effectiveness of the process: it can be seen that, the larger is the reduction of volatile solids, the more efficient is the conversion into biogas, i.e. the larger is the amount of generated gas for a given volume of the digester, and the smaller is the amount of residual solid digestate that will require disposal.

The results of the tests are listed in Table I, and are shown in the chart of FIG. 3.

TABLE I
(Conventional pulped BMW)

| Pilot | Dilution % | RV % |
|---|---|---|
| 1 | 4 | 43 |
| 2 | 4 | 50 |
| 1 | 8 | 41 |
| 2 | 8 | 39 |
| 1 | 10 | 42 |
| 2 | 10 | 38 |

It can be seen that the values of RV % obtained in the several tests with pulped BMW are in the range from about 40% to 50%, consistent with data generally found in the literature.

Six further tests were run in the same two pilot plants and in the same conditions, except that the digester was fed with gelled BMW as disclosed above with reference to FIG. 1. The BMW had been extruded through a grid having 10 mm-bore holes, under a pressure of 50 bar, and had the appearance of a doughy, jamlike gel. The gelled BMW was diluted with water to such a degree that the total solid (ST) were 4%, 8% and 10% in weight in three successive pairs of tests. The dwelling times for these tests were also maintained at 25 days, at a temperature of 40° C. The generated biogas was measured in gas meter 28, and the unloaded digestate was each time weighed.

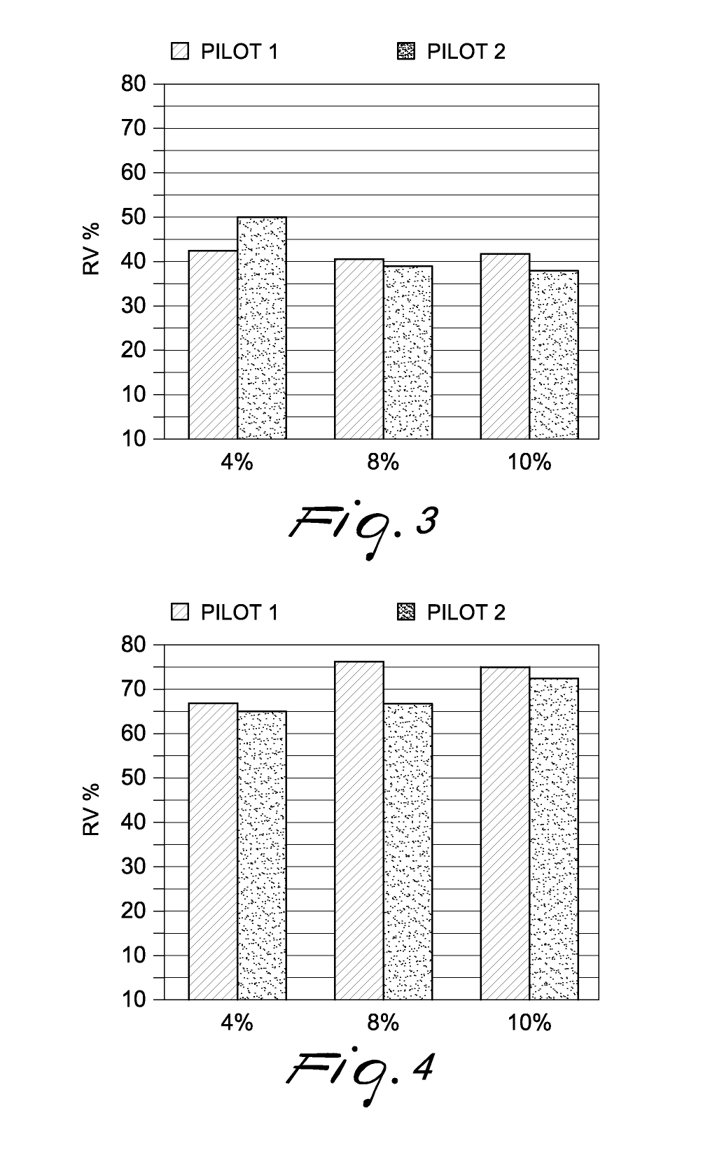

The results of the above tests are listed in Table II, and are also shown in the chart of FIG. 4.

TABLE II
(Extruded BMW according to invention)

| Pilot | Dilution % | RV % |
|---|---|---|
| 1 | 4 | 67 |
| 2 | 4 | 65 |
| 1 | 8 | 77 |
| 2 | 8 | 67 |
| 1 | 10 | 75 |
| 2 | 10 | 73 |

In this case, the values of RV % in the several tests made with gelified BMW, according to the teachings of the invention, are found in the range 65% to 75%, about at least 20-25 points above the values of Table I, on the average.

It can be seen that the method according to conventional technology attains its maximum efficiency at a dilution of 4%, and the efficiency drops for a dilution of 8% and even more for 10%, presumably because as high a dilution as feasible is required in order to facilitate mixing. By contrast, use of gelified BMW according to the invention not only tolerates a lower dilution, but in fact attains the best efficiency at a 10% dilution.

It can be seen from the above tables and charts that the method of the invention has a number of advantages in several respects. From an economic standpoint, the method achieves a larger production of biogas while decreasing the amount of neutralized solids and consequently the expense required for their disposal. From another point of view, the method is advantageous in the protection of the environment, since a greater consumption of biogas entails a lower consumption of fossil fuel.

Furthermore, since the organic material is not whipped or thrashed at high speed as in the prior art, but rather is gradually squeezed under semi-static conditions, there is much less tendency for inert materials to be ground down to fine particles; any crumbs of inert material, such as stone, glass, plastic, tend to be macroscopic, and readily separate from the main organic material to sink to the bottom or float to the surface. Accordingly, there are little or no fine particles which may be liable to be englobed into the biodegradable gel material, to become free later, as the clots are dissolved. Consequently, both the sediments and the hat grow more slowly, thus allowing longer operating periods between shutdowns for cleaning.

Although water has been used as a dilution fluid in all tests described above, i.e. both in the tests using the conventional pre-treatment and in tests using the pre-treatment of the invention, it will be obvious to persons skilled in the art that other liquids can be used, such as whey, sewage water and other similar liquids.

The disclosures in Italian Patent Application No. TO2012A000456 from which this application claims priority are incorporated herein by reference.

What is claimed is:

1. A process for the generation of fuel gas from solid waste containing an organic fraction comprising biological cells enclosed by cellular membranes, the process including the following steps:
squeezing the solid waste against a grid of small-bore holes, under a pressure higher than the bursting pressure of the cells, whereby said organic fraction is extruded through the holes to form a gel of a doughy consistency in which most of the cellular membranes are disrupted; and
subjecting said extruded gel to anaerobic fermentation in a biodigester to produce fuel gas.

2. The process of claim 1, wherein said pressure of extrusion is at least 50 bar.

3. The process of claim 1, wherein the average diameter of said small-bore holes is less than 12 mm.

4. The process of claim 1, wherein the average diameter of said small-bore holes is less than 10 mm.

5. The process of claim 1, wherein the average diameter of said small-bore holes is about 8 mm.

6. The process of claim 1, wherein said gel is diluted with an aqueous fluid before being loaded into the biodigester.

7. The process of claim 6, wherein said gel is diluted with said aqueous fluid to such a degree that the total solid is at least 8% on the total weight.

8. The process of claim 7, wherein the gel is diluted with said aqueous fluid to such a degree that the total solid is about 10% on the total weight.
